(19) United States
(12) Patent Application Publication
(10) Pub. No.: US 2026/0251522 A1
(43) Pub. Date: Aug. 27, 2026

(54) METHOD FOR MEASURING THE AMBIENT CONCENTRATION OF A LIGHT GAS WITH A MASS-SPECTROMETRIC COUNTERFLOW LEAK DETECTION DEVICE

(71) Applicant: INFICON GMBH, Köln (DE)

(72) Inventor: Simon MATHIOUDAKIS, Köln (DE)

(73) Assignee: INFICON GMBH, Köln (DE)

(21) Appl. No.: 18/874,917

(22) PCT Filed: May 25, 2023
(86) PCT No.: PCT/EP2023/064001

§ 371 Date: Dec. 13, 2024

(30) Foreign Application Priority Data
Jun. 22, 2022 (DE) 10 2022 115 562.5

(51) Int. Cl. G01M 3/20; G01N 33/00

(52) U.S. Cl. CPC G01M 3/205; G01N 33/005

(57) ABSTRACT

A method for determining an ambient concentration of a light gas uses a mass-spectrometric counterflow leak detection device having a mass-spectrometric gas detector, a high vacuum pump connected to the gas detector, and a pre-vacuum pump connected to the high vacuum pump. The pre-vacuum pump has a gas outlet open to the atmosphere. The pre-vacuum pump is used to evacuate the high vacuum pump and/or the gas detector. Air from the environment of the pre-vacuum pump is admitted into the running pre-vacuum pump or into its inlet-side pre-vacuum area in such a way that a gas component of the admitted air/gas mixture, which is lighter than air, is introduced in counterflow into the gas detector and is detected there, while the remaining gas components of the introduced air mixture are delivered to the surrounding atmosphere through the outlet of the pre-vacuum pump.

### CROSS-REFERENCE TO RELATED APPLICATIONS

The present application is a national stage application filed under 35 U.S.C. § 371 of PCT Application No. PCT/EP2023/064001, filed on May 25, 2023, which claims priority to German patent application DE 102022115562.5, filed on Jun. 22, 2022, the entire contents of all of which are incorporated by reference herein.

### BACKGROUND OF THE DISCLOSURE

### 1. Field of the Disclosure

The disclosure relates to a method for detecting light gas components in the outer environment of a mass-spectrometric counterflow leak detector.

### 2. Description of Related Art

Mass-spectrometric counterflow leak detection devices typically comprise: a port to a test specimen to be examined or to a test chamber into which the test specimen to be examined is placed; a gas detector connected to the port in the form of a mass spectrometer; a high vacuum pump, usually in the form of a turbomolecular pump, the inlet of which is connected to the mass spectrometer, the outlet of which is connected to the inlet of a pre-vacuum pump, the outlet of which delivers the evacuated gas into the surrounding atmosphere. In addition, the high vacuum pump typically comprises an intermediate gas inlet connected to the test specimen port or the test chamber port, respectively. Additionally, the pre-vacuum pump can be connected directly to the mass spectrometer via a bypass line bridging the high vacuum pump for massive leak detection. The test specimen port or the test chamber port, respectively, can be connected to the inlet of the pre-vacuum pump via an additional booster pump, wherein the booster pump can be connected to the intermediate gas inlet of the high vacuum pump.

For leak detection, the test chamber port and the test chamber connected thereto are first evacuated with the pre-vacuum pump and the booster pump, if present. Moreover, the pre-vacuum pump generates the required pre-vacuum at the outlet of the high vacuum pump, so that the high vacuum pump evacuates the mass spectrometer. As soon as the vacuum required to operate the mass spectrometer is reached at the inlet of the high vacuum pump, a connecting valve in the connection between the test chamber port and the high vacuum pump is opened, causing gas to flow from the test chamber into the high vacuum pump and to pass in counterflow through the high vacuum pump into the mass spectrometer for analysis. In the case of a leaking test specimen, gas from inside the test specimen enters the test chamber and from there enters the mass spectrometer in counterflow and can be detected there.

For mass-spectrometric counterflow leak detection, it is often of interest to determine the concentration of certain light gas components of the ambient air. Light gas components are gas components of the air mixture from the ambient air that are lighter than air. One such light gas component can be helium, for example.

Conventionally, ambient helium concentration is determined using separate gas analyzers. Alternatively, in a mass-spectrometric counterflow leak detector, it is possible to switch off the pre-vacuum pump and open a gas ballast valve of the pre-vacuum pump to vent it and thereby slow it down. As soon as the pre-vacuum pump stops, gas can be introduced into the mass spectrometer via the gas ballast valve and a bypass line bridging the high vacuum pump for massive leak detection and can be measured there. The bypass line is a direct connection line between the inlet of the pre-vacuum pump and the mass spectrometer. Typically, such bypass lines are provided with an additional throttle for massive leak detection.

### SUMMARY OF THE DISCLOSURE

The object of the disclosure is to provide an improved method for determining the concentration of a light gas component in the environment of a mass-spectrometric counterflow leak detector.

The disclosure relates to a mass-spectrometric counterflow leak detection device having a pre-vacuum pump, a high vacuum pump, and a mass-spectrometric gas detector. The outlet of the pre-vacuum pump can be open to the atmosphere or, for example, can be connected to an exhaust line that discharges the pumped gases to the atmosphere. The inlet of the pre-vacuum pump is connected to an outlet of the high vacuum pump. The inlet of the high vacuum pump is connected to the mass spectrometer. An intermediate gas inlet of the high vacuum pump is connected to the outlet via a separate connection line for a vacuum test chamber and/or a test specimen. In addition, the port for the test chamber or the test specimen, respectively, could be connected to the inlet of the pre-vacuum pump via an additional booster pump, which, however, is not absolutely necessary. It would be conceivable for an intermediate gas outlet of the booster pump to be connected to the intermediate gas inlet of the high vacuum pump.

The high vacuum pump is typically a turbomolecular pump. The pre-vacuum pump is provided with a gas ballast that opens into the outside atmosphere or opens into a fresh air line. The gas ballast can comprise a selectively switchable valve and thus be selectively opened or closed.

For mass-spectrometric counterflow leak detection, the pre-vacuum pump is conventionally used to generate the pre-vacuum pressure at the outlet of the high vacuum pump, so that the high vacuum pump can generate the required high vacuum inside the mass spectrometer. Via the intermediate gas inlet of the high vacuum pump, gas from the test chamber or from the test specimen, respectively, is admitted in counterflow through the high vacuum pump into the mass spectrometer and analyzed there. In doing so, it is determined whether the analyzed gas has a gas component that indicates a leak in the test specimen.

In order to meanwhile detect or analyze light gas components, which are lighter than air, from the outer environment of the pre-vacuum pump, the gas ballast valve can be opened or air from the outer environment of the pre-vacuum pump can be admitted into the pre-vacuum pump through the open gas ballast, so that at least one light gas component, such as helium, flows out in counterflow through the pre-vacuum pump through its inlet and from there into the mass spectrometer, while the remaining gas components are delivered to the environment through the outlet of the pre-vacuum pump. Alternatively or additionally, an air inlet can open directly into the pre-vacuum area of the pre-vacuum pump, i.e., for example, into the connection line between the pre-vacuum pump and the high vacuum pump and/or into the bypass line described further below for bypassing the high vacuum pump.

Thus, the disclosure has the advantage that the analysis of the ambient air and, in particular, the measurement of concentrations of light gas components of the ambient air can be carried out during the actual leak detection with the mass spectrometer without having to interrupt the leak detection and/or switch off the pre-vacuum pump. In this respect, the light gas component to be measured can be introduced in counterflow from the inlet of the pre-vacuum pump to the outlet of the high vacuum pump, through the high vacuum pump to its inlet, and from there into the mass spectrometer. Alternatively or additionally, the light gas component to be measured can enter the mass spectrometer through a bypass line connecting the inlet of the pre-vacuum pump to the mass spectrometer for mass leak detection, which bypass line bridges the high vacuum pump. The bypass line can be provided with an additional throttle. Such a bypass line enables the detection of particularly large leaks before the high vacuum pump in the mass spectrometer has generated the required high vacuum pressure.

The light gas component then flows against the pumping direction from the gas ballast through the pre-vacuum pump towards the mass-spectrometric gas detector. The pressure at the outlet of the high vacuum pump depends only on the speed of the pre-vacuum pump and not on the proportion of the light gas component (e.g. helium) to be measured in the ambient air. The measured deviation in the leak rate signal or ion raw current signal of the mass spectrometer depends on the ambient helium concentration or on the concentration of the light gas component in the ambient air, respectively.

Light gases, such as helium, flow in the direction of the mass spectrometer against the actual pumping direction. The lower the speed of the pre-vacuum pump, the more helium flows through the pre-vacuum pump in counterflow through its inlet back into the mass spectrometer. The increase in the light gas component is greater than the total pressure increase in the mass spectrometer due to the different compressions. By changing or modulating the speed of the pre-vacuum pump, the ambient concentration of light gases, such as helium, can thus be determined even without actuating a valve.

The determination of the ambient concentration of the light gas component can be carried out during the actual leak detection or also following a leak detection of a test specimen without having to switch off the pumps. The pre-vacuum pump and the high vacuum pump continue to run so that the time needed to shut down and then re-start the vacuum pumps is eliminated.

### BRIEF DESCRIPTION OF THE DRAWINGS

In the following, exemplary embodiments of the disclosure are explained in more detail with reference to the Figures.

FIG. 1 is a first exemplary embodiment.

FIG. 2 is a second exemplary embodiment.

### DETAILED DESCRIPTION OF THE DISCLOSURE

A mass-spectrometric counterflow leak detection device 10 comprising a gas detector 12 in the form of a mass spectrometer is illustrated. Gas detector 12 is connected to inlet 14 of a high vacuum pump 16 in the form of a turbomolecular pump in a gas-conducting manner. Outlet 18 of high vacuum pump 16 is connected to inlet 22 of a pre-vacuum pump 24 in a gas-conducting manner via a connection line provided with a selectively controllable valve 20. Outlet 26 of pre-vacuum pump 24 is open to the atmosphere or opens into an exhaust line.

In addition, inlet 22 of pre-vacuum pump 24 is connected to mass spectrometer 12 via a separate bypass line 28. Bypass line 28 bridges high vacuum pump 16 and the controllable valve 20 and is provided with a throttle 30 in order to thus enable a massive leak detection.

Leak detection device 10 is further provided with a port 32 for a test chamber or a test specimen. In the illustrated exemplary embodiment, a test chamber 35 is vacuum-connected to port 32. Test chamber 35 receives a test specimen to be examined for leaks and is then evacuated. For this purpose, port 32 is connected to a booster pump 34 in a gas-conducting manner. In this respect, inlet 36 of the booster pump is connected to port 32 in a gas-conducting manner, while outlet 38 of the booster pump is connected to inlet 22 of the pre-vacuum pump via a gas line 42 provided with a further valve 40.

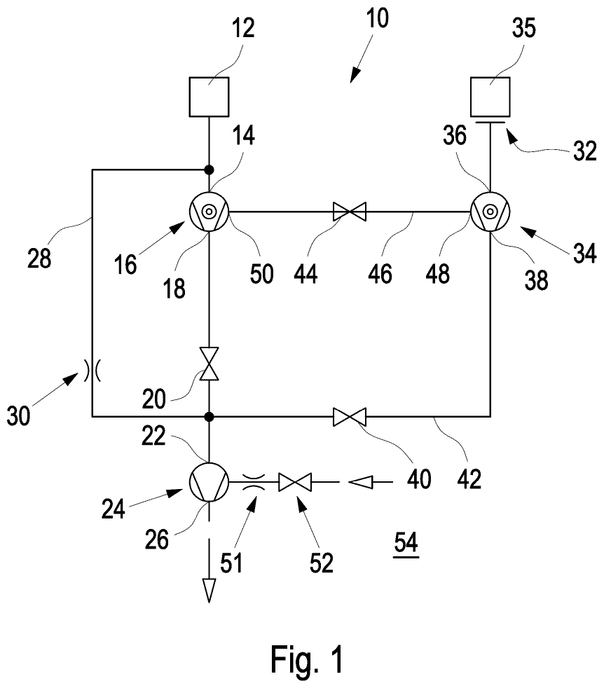

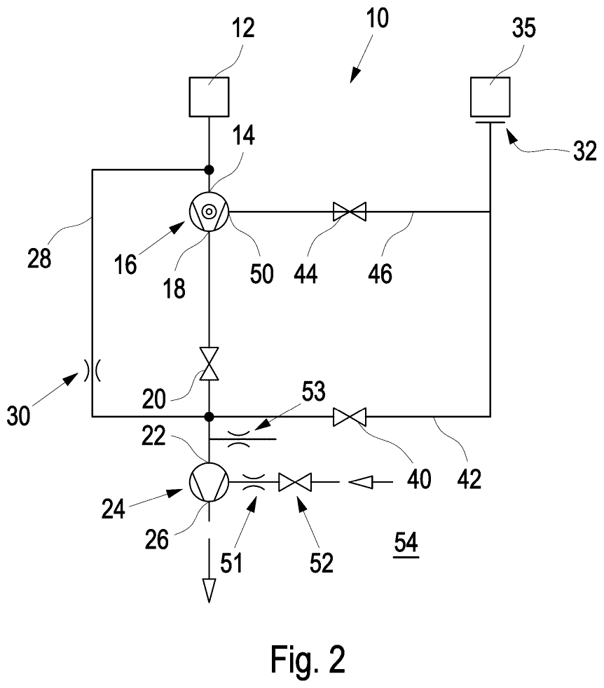

Booster pump 34 is not essential to the disclosure. FIG. 2 shows an exemplary embodiment without booster pump. The exemplary embodiment of FIG. 2 corresponds to that of FIG. 1 except for the missing booster pump 34 and air inlet 53.

An intermediate gas line 46 comprising a further selectively operable valve 44 connects an intermediate gas outlet 48 of booster pump 34 to an intermediate gas inlet 50 of high vacuum pump 16.

Pre-vacuum pump 24 is also provided with a gas ballast valve 52, formed separately from inlet 22 and outlet 26, which connects the inside of pre-vacuum pump 24 to outer environment 54 of pre-vacuum pump 24 and leak detection device 10. Gas ballast valve 52 is selectively operable and can be opened and closed.

A throttle point 51 is provided between gas ballast valve 52 and pre-vacuum pump 24 to determine the gas flow admitted to the pre-vacuum pump 24 through the gas ballast. According to the disclosure, gas ballast valve 52 can be omitted. In this case, only throttle point 51 is provided at the inlet forming the gas ballast of pre-vacuum pump 24. Throttle point 51 can be enabled, for example, by selecting a suitable cross-section of the gas ballast inlet.

As an alternative or in addition to the inlet of air from environment 54 via the gas ballast of pre-vacuum pump 24, the ambient air can also be admitted to the pre-vacuum area of pre-vacuum pump 24 via an air inlet 53 as shown in FIG. 2. In FIG. 2, as an exemplary embodiment, an air inlet 53 is provided, which can be open to the atmosphere or connected to a fresh air line, and which opens into the connection line between inlet 22 and valve 20, i.e. into the connection line connecting pre-vacuum pump 24 to high vacuum pump 16. Air inlet 35 is shown in FIG. 2 in addition to the inlet via the gas ballast of pre-vacuum pump 24, but can also be provided as an alternative without the gas ballast. In this case, the gas ballast on the pre-vacuum pump 24 is omitted. Air inlet 53 is also provided with a throttle similar to throttle point 51 of the gas ballast. The same is also possible with the exemplary embodiment in FIG. 1.

During operation, test chamber 35 is evacuated with the aid of pre-vacuum pump 24 and booster pump 34. Pre-vacuum pump 24 generates the pre-vacuum required at the outlet of high vacuum pump 16, into which high vacuum pump 16 evacuates the content of mass spectrometer 12. Meanwhile, pre-vacuum pump 24 also directly evacuates mass spectrometer 12 via bypass line 28.

As soon as the vacuum pressure required for the operation of mass spectrometer 12 is reached, valve 44 is opened and valve 40 is closed, so that test gas and/or leakage gas from the inside of test chamber 35 enters mass spectrometer 12 through booster pump 34, intermediate gas line 36 and in counterflow through high vacuum pump 16 and can be analyzed there.

In order to be able to determine the gas concentration of the helium content in the air of environment 54 in a quick and simple manner, gas ballast valve 52 is opened, while pre-vacuum pump 24 is still running. In the exemplary embodiment without gas ballast valve, gas ballast 52 is permanently open and can be connected to a fresh air line. In the simplest case, gas ballast 52 can be open to the atmosphere.

In order to determine the gas concentration of the helium content of the ambient air, the speed of the vacuum pump is modulated or at least changed between two different operating states, for example, alternating between the final speed of pre-vacuum pump 24 and a reduced speed. The change in pump speed results in a change in the helium partial pressure as well as the total pressure at inlet 22 of pre-vacuum pump 24. In the measurement signal of mass spectrometer 12, a change or a signal deviation can then be measured for the helium content, because the helium from pre-vacuum pump 24 enters mass spectrometer 12 either via bypass 28 or in counterflow through high vacuum pump 16. The supply of air flowing into pre-vacuum pump 24 from environment 54 can be influenced by opening and closing gas ballast valve 52 in the case of a gas ballast valve 52.

Gas components of the ambient air that are lighter than the average value of the gas components of the ambient air will flow out of pre-vacuum pump in counterflow through inlet 22 and from there flow in counterflow through high vacuum pump 16 and/or through bypass line 28 into the high vacuum of mass spectrometer 12, where they can be measured. On the contrary, gas components of the ambient air that are heavier than the ambient air are returned to environment 54 via outlet 26 of pre-vacuum pump 24. In this way, helium from ambient air 54 enters mass spectrometer 12 and can be analyzed there without having to shut down pre-vacuum pump 24, high vacuum pump 16, and/or booster pump 34.

As an alternative or in addition to the gas ballast of pre-vacuum pump 24, an air inlet can be provided for supplying ambient air directly into the pre-vacuum area of pre-vacuum pump 24, for example as an air inlet 53 into the connection line between pre-vacuum pump 24 and high vacuum pump 16, between inlet 22 and valve 20, and/or into bypass line 28.

1-10. (canceled)

11. A method for determining an ambient concentration of a light gas with a mass-spectrometric counterflow leak detection device having a mass-spectrometric gas detector, a high vacuum pump connected to the mass-spectrometric gas detector, and a pre-vacuum pump connected to the high vacuum pump, wherein the pre-vacuum pump comprises a gas outlet open to the atmosphere, wherein the pre-vacuum pump is used to evacuate the high vacuum pump and/or the gas detector, the method comprising:
admitting air from the environment of the pre-vacuum pump into the pre-vacuum pump that is running or into an inlet-side pre-vacuum area in such a way that a gas component lighter than air of the admitted air is introduced in counterflow into the gas detector and is detected by the gas detector while the remaining gas components of the admitted air are delivered to the surrounding atmosphere through the outlet of the pre-vacuum pump.

12. The method according to claim 11, wherein the gas component lighter than air is selected from the group consisting of: neon, helium, hydrogen, and deuterium.

13. The method according to claim 11, wherein the gas component lighter than air is introduced into the gas detector in counterflow through the high vacuum pump or through a bypass line bridging the high vacuum pump and connecting the pre-vacuum pump to the gas detector for massive leak detection.

14. The method according to claim 12, wherein the gas component lighter than air is introduced into the gas detector in counterflow through the high vacuum pump or through a bypass line bridging the high vacuum pump and connecting the pre-vacuum pump to the gas detector for massive leak detection.

15. The method according to claim 11, wherein the pre-vacuum pump comprises an open gas ballast through which the air from the environment of the pre-vacuum pump is admitted into the running pre-vacuum pump.

16. The method according to claim 11, wherein the inlet-side pre-vacuum area has a separate air inlet provided upstream of the inlet of the pre-vacuum pump through which the air from the environment of the pre-vacuum pump is admitted into the inlet-side pre-vacuum area thereof.

17. The method according to claim 11, further comprising:
determining the ambient concentration of the gas component lighter than air of the admitted air by changing a speed of the pre-vacuum pump; and
determining a proportion of the gas component lighter than air from a resulting change in a measurement signal of the mass-spectrometric gas detector.

18. The method according to claim 11, further comprising:
changing a speed of the pre-vacuum pump from a first speed value to at least a second speed value different than the first speed value; and
evaluating a response of a measurement signal representing a partial pressure of the gas component lighter than air.

19. The method according to claim 11, wherein the pre-vacuum pump is a roots pump and/or a claw pump.

20. The method according to claim 11, further comprising:
analyzing the gas component lighter than air admitted from the environment of the pre-vacuum pump with the mass-spectrometric gas detector during the detection by the mass-spectrometric gas detector.

21. The method according to claim 11, wherein the pre-vacuum pump is not deactivated during a measurement of the gas component lighter than air from the environment of the pre-vacuum pump.
